(19) United States
(12) Patent Application Publication
(10) Pub. No.: US 2005/0112599 A1
(43) Pub. Date: May 26, 2005

(54) Screening of expression profile of fat specific genes expressed by growing stages in swine and functional cDNA chip prepared by using the same

(76) Inventors: Chul-Wook Kim, Gyeongsangnam-do (KR); Jung-Sou Yeo, Daegu (KR); Jung-Gyu Lee, Gyeongsangnam-do (KR); Young-Min Song, Gyeongsangnam-do (KR); Kwang-Keun Cho, Seoul (KR); Ki-Hwa Chung, Gyeongsangnam-do (KR); Il-Suk Kim, Gyeonggi-do (KR); Sang-Keun Jin, Gyeongsangnam-do (KR); Su-Hyun Park, Gyeongsangnam-do (KR); Ji-Won Jung, Gyeongsangnam-do (KR); Min-Jung Lee, Gyeongsangnam-do (KR); Eun-Jung Kwon, Gyeongsangnam-do (KR); Eun-Segk Cho, Gyeongsangnam-do (KR); Hwok-Rai Cho, Gyeongsangnam-do (KR); Sun-Min Shin, Gyeongsangnam-do (KR); Hee-Sun Nam, Gyeongsangnam-do (KR); Yeon-Hee Hong, Gyeongsangnam-do (KR); Sung-Kwang Hong, Gyeongsangnam-do (KR); Yang-Su Kang, Gyeongsangnam-do (KR); Young-Joo Ha, Gyeongsangnam-do (KR); Jeong-Man Rou, Gyeongsangnam-do (KR); Suk-Chun Kwack, Gyeongsangnam-do (KR); In-Ho Choi, Seoul (KR); Byeong-Woo Kim, Gyeongsangnam-do (KR)

Correspondence Address: OSTROLENK FABER GERB & SOFFEN, 1180 AVENUE OF THE AMERICAS, NEW YORK, NY, 100368403, US

(21) Appl. No.: 10/788,562

(22) Filed: Feb. 27, 2004

(30) Foreign Application Priority Data
Nov. 24, 2003 (KR) 2003-83654

(51) Int. Cl. C12Q 1/68; G06F 19/00; G01N 33/48; G01N 33/50; C12M 1/34

(52) U.S. Cl. 435/6; 435/287.2; 702/20

(57) ABSTRACT

The present invention relates to screening of the expression profile of fat specific genes according to the growing stages in swine and a functional cDNA chip using the same and provides expression files of the fat specific genes specifically expressed according to the growing stages in the muscle and fat tissues of swine. Also, the present invention provides a functional cDNA chip for meat quality evaluation and screening of specific genes in swine prepared by integrating only the fat specific genes screened as described above. Therefore, the functional cDNA chip can be used to evaluate of meat quality according to breeds of swine and to bring a high meat quality swine.

### TECHNICAL FIELD

The present invention relates to screening of expression profile of fat specific genes according to growing stages of swine and a functional cDNA chip using the same. More particularly, the present invention relates to screening of expression profile of fat specific genes specifically expressed in the muscle and fat tissues of swine according to the growing stages and a functional cDNA chip for evaluating high meat quality and screening of specific genes of swine prepared by integrating only the fat specific genes.

### BACKGROUND ART

Since native black swine has a thick back fat layer and shows a low growth rate and a low production rate, the pig farmers do not prefer to raise it. However, this swine has solid fat tissue, white fat color, excellent texture, abundant and sweet gravy, which suits our taste and thus, its consumption is recently tending to increase. However, genetic research of the native swine, preservation and control of pedigree, analysis of meat quality related genes are still insufficient. Particularly, the meat quality related genetic traits are composite results of more genetic traits, as compared to the meat quantity related traits and research on this has not been much conducted (Cameron, 1993).

Important genes affecting meat quality in swine which have been known to so far include ryanodine receptor gene (RYR) resulting in PSE (pale, soft, exudative) pork meat (Eikelenboom and Minkema, 1974; Smith and Bampton, 1977; Webb, 1981; Christian and Mabry, 1989; Fujii el al., 1991) and acid meat genes (Rendement Napole, Le Roy el al., 1990; Lundstrom el al., 1996). In addition, by QTL (quantitative trait loci) analysis, meat quality related regions or various candidate genes are known. Swine leucocyte antigen (SLA) composite existing in No. 7 chromosome (Geffrotin el al., 1984) and micorsatellite marker S0064, S0066, S0102 or TNF around this region are known to be associated with back fat thickness, sirloin unit area, meat quality traits, boar taint (Jung el al., 1989; Rothschild el al., 1995; Bidanel el al., 1996). Also, it has been found that back fat thickness- and abdominal fat content-related QTL is present in positions of microsatellite marker S0001 to S0175 (Andersson el al., 1994). Further, it has been reported that the pituitary-specific transcription factor (PIT1) gene which is known as a regulation factor of hormones (Yu el al., 1995). The intramuscular fat content (IMF) is known to largely affect the tenderness, juiciness and taste of meat (Devol el al., 1988; Cameron, 1990). H-FAPB (heart-fatty acid binding protein) has been reported as a gene which exerts influence on the intramuscular fat content (Gerbens el al., 1997). The Microsatellite SW1823 to S0003 (74 to 79cM) positions existing in No. 6 chromosome has been studied on the relation of such properties of meat (Grindflek el al., 2001).

Thus, as QTL affecting meat quality traits was largely found in NO. 4, 6 and 7 chromosomes (Clamp el al., 1992; Andersson el al., 1994; Renard el al., 1996; Rohrer and Keele 1998a, 1998b; Wang el al., 1998; de Koning el al., 1999; Ovilo el al., 2000; Gerbens el al., 2000), much research has been conducted to develop a meat quality related marker centering around these chromosome.

For last few years, there have been efforts to develop a gene map comprising anonymous meat quality-related gene markers of swine and known markers. Up to now, several technologies to analyze gene expression at the mRNA level such as northern blotting, differential display, sequential analysis of gene expression and dot blot analysis have been used to examine the genetic difference in swine. However, these methods have disadvantages which are not suitable for simultaneous analysis of a plurality of expressed products. In recent, a new technology such as cDNA microarray to overcome such disadvantages has been developed. The cDNA microarray becomes one of the strongest means to study gene expression in various living bodies. This technology is applied to simultaneous expression of numerous genes and discovery of genes in a large scale, as well as polymorphism screening and mapping of genetic DNA clone. It is a highly advanced RNA expression analysis technology to quantitatively analyze RNA transcribed from already know or not-known genes.

DNA chip types which are currently used include composite DNA chips constructed by designing a primer based and combining genes from cDNA library on the data base information and functional DNA chips constructed by combining related genes based on the existing references. When the composite DNA chip is used for translation, there is difficulty in translation due to the action of non-related genes and enormous efforts are required to finally interpret the biological roles. Also, since it is based on the database, there may be difficulties due to a new gene without information or possibility of partial absence of related gene. Meanwhile, the functional DNA chip is easy to be translated but requires another collection of genes for characterization of genes which are not described in the existing references or not-know for their functions. Therefore, the DNA construction on a chip is very important for effective interpretation.

Considering these matters, the present inventors have introduced the cDNA microarray technology into screening of the expression profile of genes related to meat quality in a specific tissue of swine and made a functional cDNA chip by integrating only the specific gene identified from the screening which would be applied to swine improvement with high meat quality and evaluation of meat quality according to breeds and tissues of swine.

### DISCLOSURE OF INVENTION

Therefore, an object of the present invention is to screen an expression profile of specific genes differentially expressed according to growing stages of the fat by hybridizing a substrate integrated with a probe prepared from total RNA isolated from the muscle and fat tissues of swine with a target DNA from the muscle and fat tissues of swine.

It is another object of the present invention to provide a functional cDNA chip for meat quality evaluation and screening of specific genes in swine, which is prepared by integrating only the specific genes obtained from the screening.

According to the present invention, the above-described objects are accomplished by preparing thousands of ESTs from total RNA isolated from the muscle and fat tissues of swine by PCR, cloning them to analyze and screen their nucleotide sequences in the database, amplifying the ESTs by PCR, followed isolation and purification, arraying the product with a control group on a slide using a DNA chip array, preparing a target DNA from total RNA isolated from the muscle and fat tissues of swine to screen an expression profile of a growth-related gene, hybridizing the slide (probe DNA) with the target DNA, scanning the product to obtain an image file, examining the expression aspect of the fat-related gene differentially expressed according to the growing stages of swine based on the image file, and preparing a functional cDNA chip by integrating only the fat specific genes of swine according to the growing stages.

The present invention comprises the steps of preparation of ESTs from muscle and fat tissues of swine and identification of sequence information; preparation of a probe DNA using the ESTs; hybridization of a fluorescent-labeled target DNA (ESTs) from the muscle and fat tissues of swine with the probe DNA, followed by scanning and analysis of an image file; examination of the expression profile of a fat-related genes according to growing stages in swine; and preparing a functional cDNA by integrating only the fat specific gene.

The functional cDNA chip for meat quality evaluation and screening of specific genes in swine is prepared by the following steps: preparing 4434 ESTs from total RNA isolated from the muscle and fat tissues of swine by PCR; arraying the ESTs with an enzyme control on a slide using a DNA chip array; preparing a target DNA having 3-dCTP or 5-dCTP bound from total RNA isolated from the muscle and fat tissues of swine; hybridizing the slide (probe DNA) with the target DNA, scanning the product and analyzing the image file to examine the expression aspect of the fat-related genes specifically expressed according to the growing stages in swine; and preparing a functional cDNA chip by integrating only the screened fat specific gene according to the growing stages in swine.

The functional cDNA chip for meat quality evaluation and screening of specific genes in swine according to the present invention comprises a probe comprising fat specific genes specifically expressed in the muscle and fat tissues of swine and a substrate on which the probe is immobilized.

The probe DNA immobilized on a DNA microarray of the functional cDNA chip for meat quality evaluation and screening of specific genes in swine according to the present invention includes collagen, firbronectin, inhibitor of metalloproteinase 3 and integrin β-1 subunit.

The substrate of the functional cDNA chip according to the present invention is preferably a polymer film such as silicone wafer, glass, polycarbonate, membrane, polystyrene or polyurethane. The DNA microarray according to the present invention may be prepared by immobilizing a probe on a substrate by a conventional method for preparing a DNA microarray, including photolithography, piezoelectric printing, micro pipetting, spotting and the like. In the present invention, the spotting method is used.

The kit for meat quality evaluation and screening of specific genes in swine comprises the functional cDNA chip having the fat specific genes according to the growing stages in swine integrated, Cy5-dCTP or Cy3-dCTP bound cDNA from RNA of the tissue to be screened, a fluorescence scanning system and computer analysis system.

### BEST MODE FOR CARRYING OUT THE INVENTION

Now, the concrete construction of the present invention will be explained through the following Examples. However, the present invention is not limited thereto.

### EXAMPLE

### Example 1

### Screening of Expression Profile of Fat Specific Genes According to the Growing Stages in Swine

In order to screen the expression profile of fat specific genes specifically expressed according to the growing stages in swine, a probe DNA was prepared from total RNA isolated from muscle and fat tissues of Kagoshima Berkshire and the total RNA of the tissues was fluorescently labeled to prepare a target DNA. These DNAs are hybridized and scanned. The resulting image file was analyzed to screen the fat specific genes according to the growing stages in swine.

### Preparation Example 1

### Preparation and Array of Probe DNA

Firstly, probe DNA, which was cDNA amplified by PCR, was prepared and attached to a slide glass. Total RNA was extracted from the muscle and fat tissues of the longissimus dorsi of Kagoshima Berkshire (body weight of 30 kg and 90 kg) using a RNA extraction kit (Qiagen, Germany) according to the manual and mRNA was extracted using an oligo (dT) column. The extracted mRNA sample was subjected to RT-PCR using SP6, T3 forward primer, T7 reverse primer (Amersham Pharmacia Biotech, England) to synthesize cDNA. The total volume of each PCR reactant was 100 μl. 100 pM of forward primer and reverse primer were each transferred to a 96-well PCR plate (Genetics, England). Each well contained 2.5 mM dNTP, 10×PCR buffer, 25 mM MgCl₂, 0.2 μg of DNA template, 2.5 units of Taq polymerase. PCR was performed in GeneAmp PCR system 5700 (AB Applied BioSystem, Canada) under the following conditions: total 30 cycles of 30 seconds at 94° C., 45 seconds at 58° C., 1 minute at 72° C.

The size of the amplified DNA was identified by agarose gel electrophoresis. The PCR product was precipitated with ethanol in 96-well plate, dried and stored at −20° C. Total 4434 cDNAs (ESTs), prepared as described above, were cloned to analyze nucleotide sequences of genes which swine has and their genetic information was identified from the database at NCBI. The genes having information were isolated and purified by PCR. The enetic locus and map for the total 4434 cDNAs (ESTs) were constructed. The total 4434 cDNAs (ESTs) and 300 yeast controls were arrayed in an area of 1.7 cm². Then, the probe DNA was spotted on a slide glass for microscope (produced by Corning), coated with CMT-GAPS™ aminosilane using Microgrid II (Biorobotics). The probe DNA was printed onto Microgrid II using a split pin. The pin apparatus was approached to the well in the microplate to inject the solution into the slide glass (1 to 2 nL). After printing of the probe DNA, the slide was dried and the spotted DNA and the slide were UV cross-linked at 90 mJ using Stratalinker™ (Stratagene, USA), washed twice with 0.2% SDS at room temperature for 2 minutes and washed once with third distilled water at room temperature for 2 minutes. After washing, the slide was dipped in a water tank at 95° C. for 2 minutes and was blocked for 15 minutes by adding a blocking solution (a mixture of 1.0 g NaBH₄ dissolved in 300 mL of pH7.4 phosphate buffer and 100 mL of anhydrous ethanol). Then, the slide was washed three times with 0.2% SDS at room temperature for 1 minute and once with third distilled water at room temperature for 2 minutes and dried in the air.

### Preparation Example 2

### Preparation of Target DNA and Hybridization

In order to prepare a target DNA to screen the fat specific genes specifically expressed in the muscle and fat tissues of swine, the muscle tissue on the longissimus dorsi area was taken from the Kagoshima Berkshires having body weights of 30 kg and 90 kg. The fat tissue was taken from the Kagoshima Berkshire having a body weight of 30 kg. The muscle and fat tissues were cut into 5˜8 mm length, frozen with liquid nitrogen and stored at −70° C.

Total RNAs were isolated from 0.2 to 1.0 g of the experimental group and the control group according to the manual of Trizol™ kit (Life Technologies, Inc.) to prepare the target DNA. Trizol™ was added to the tissue in an amount of 1 mL of Trizol™ per 50 to 100 mg of tissue and disrupted using a glass-Teflon or Polytron homogenizer. The disrupted granules were centrifuged at 4° C. at a speed of 12,000 g for 10 minutes and 1 mL of the supernatant was aliquoted. 200 μl of chloroform was added to each aliquot, voltexed for 15 seconds, placed on ice for 15 minutes and centrifuged at 4° C. at a speed of 12,000 g for 10 minutes. Chloroform of the same amount was again added thereto, voltexed for 15 seconds, placed on ice for 15 minutes and centrifuged at 4° C. at a speed of 12,000 g for 10 minutes. The supernatant was transferred to a new tube. 500 μl of isopropanol was added to the tube, voltexed and placed on ice for 15 minutes. The ice was cooled and centrifuged at 4° C. at a speed of 12,000 g for 5 minutes. The supernatant was removed, mixed with 1 mL of 75% cold ethanol and centrifuged at 4° C. at a speed of 12,000 g for 5 minutes. The supernatant was removed, freeze-dried on a clean bench for 30 minutes and take into 20 μl of RNase-free water or DEPC water to dissolve RNA. The total DNA concentration was set to 40 μg/17 μl for electrophoresis.

The target DNA was prepared according to the standard first-strand cDNA synthesis. Briefly, according to the method described by Schuler (1996), 40 μg of total RNA and oligo dT-18mer primer (Invitrogen Life Technologies) were mixed, heated at 65° C. for 10 minutes and cooled at 4° C. for 5 minutes. Then, 1 μl of a mixture of 25 mM DATP, dGTP and dTTP, 1 μl of 1 mM dCTP (Promega) and 2 μl of 1 mM cyanine 3-dCTP or 2 μl of 1 mM cyanine 5-dCTP, 20 units of RNase inhibitor (Invitrogen Life Technology), 100 units of M-MLV RTase, 2 μl of 10× first strand buffer were added thereto and mixed with a pipette. The reaction mixture was incubated at 38° C. for 2 hours and the non-bound nucleotide was removed by ethanol precipitation. Here, DEPC treated sterile water was used.

The slide, prepared above, was pre-hybridized with a hybridization solution (5×SSC, 0.2% SDS, 1 mg/mL herring sperm DNA) at 65° C. for 1 hour. The target DNA labeled with cyanine 3 (Cy-3) and cyanine 5 (Cy-5) was re-suspended in 20 μl of the hybridization solution at 95° C. and denatured for 2 minutes. Then, the slide were hybridized with the solution at 65° C. overnight. The hybridization was performed in a humidity chamber covered with a cover glass (Grace Bio-Lab).

After hybridization, the slide was washed 4 times with 2×SSC, 0.1% SDS at room temperature for 5 minutes while vigorously stirred in a dancing shaker. Then the slide was washed twice with 0.2×SSC for 5 minutes and 0.1×SSC for 5 minutes at room temperature.

The slid was scanned on ScanArray 5000(GSI Lumonics Version 3.1) with a pixel size of 50 μm. The target DNA labeled by cyanine 3-dCTP was scanned at 565 nm and the target DNA labeled by cyanine 5-dCTP was scanned at 670 nm. Two fluorescence intensities were standardized by linear scanning of cyanine 3-dCTP- and cyanine 5-dCTP-labeled spots. The slide was again scanned on Scanarray 4000×L with a pixel size of 10 μm. The resulting TIFF image files were analyzed on Quantarray software version 2.1 and the background was automatically subtracted. The intensity of each spot was put into Microsoft Excel from Quantarray. The results are shown in Table 1 and Table 2.

The entire gene expression pattern of ESM (early stage muscle) was compared with those of ASM (adult stage muscle) and ESF (early stage fat). The “ESM-specific” and “ASM-specific” genes are shown in Table 1 and the “ESF-specific” genes are shown in Table 2. 20 genes showed a 5 times higher expression level in ASM, as compared to ESM. Also, 18 genes showed a 10 times higher expression level in ESF, as compared to ESM, and a 5 to 10 times higher expression level in ESM, as compared to ASM.

Some of the ASM-specific genes, ESM-specific genes, ESF-specific genes including expected gene groups are shown in Table 1 and Table 2.

TABLE 1
Expression ratio of differentially expressed genes
between ESM and ASM

| | | | |
|---|---|---|---|
| | | | Ratio of |
| ESTs | Accession | | gene expression |
| No. | No.† | Description** | ESM(30)/ASM(90) |

Cellular structure and motility

| | | | |
|---|---|---|---|
| SM2149 | CAB56598 | 1-alpha dynein heavy chain | −2.1 |
| SM781 | NP_033891 | 19 kDa-interacting protein 3- | +2.1 |
| | | like | |
| SM635 | BAB19361 | Actin | +3.4 |
| SM713 | AAA51586 | Actin | +6.3 |
| SM106 | P53506 | Actin | +8.8 |
| SM1068 | AAF20165 | Actin | +5.3 |
| SM363 | B25819 | Actin | +4.3 |
| SM768 | X52815 | Actin | +3.4 |
| SMk77 | NM_001100 | Actin, alpha 1 | +15.1 |
| SM128 | NP_033740 | Actin, gamma 2 | +6.9 |
| SM902 | BC001748 | Annexin A2 | −3.2 |
| SM846 | P81287 | Annexin V | −2.8 |
| SM653 | P04272 | Annexin II | −2.2 |
| SMk340 | U75316 | Beta-myosin heavy chain mRNA | +3.0 |
| SM1605 | AAF99682 | Calpain large polypeptide L2 | +4.7 |
| SM541 | NP_000079 | Collagen | −3.2 |
| SM715 | L47641 | Collagen | −6.8 |
| SM430 | Q9XSJ7 | Collagen alpha 1 | −6.8 |
| SM758 | CGHU1S | Collagen alpha 1 | −2.1 |
| SM62 | CGHU2V | Collagen alpha 2 | −3.2 |
| SM949 | O46392 | Collagen alpha 2 | −3.3 |
| SM410 | CAA28454 | Collagen (alpha V) | −2.3 |
| SM1651 | XM_039583 | Discs, large (Drosophila) | −2.0 |
| | | homolog 5 | |
| SM1050 | AAA30521 | Fibronectin | −2.4 |
| SM491 | NM_005529 | Heparan sulfate proteoglycan 2 | −2.2 |
| SM1573 | XM_044160 | Lamin A/C | +2.6 |
| SMk55 | NP_006462 | Myosin | +3.9 |
| SMk338 | P79293 | Myosin heavy chain | +2.0 |
| SMk168 | AB025261 | Myosin heavy chain | +9.0 |
| SM1732 | NP_004678 | Myotubularin related protein 4 | +3.8 |
| SM1691 | NP_000908 | Procollagen-proline | −2.3 |
| SM690 | NP_003109 | Secreted protein, acidic | −4.4 |
| SMk173 | X66274 | Tropomyosin | +2.6 |
| SM141 | CAA38179 | Tropomyosin | +2.7 |
| SMk51 | P18342 | Tropomyosin alpha chain | +9.6 |
| SM1043 | P06469 | Tropomyosin alpha chain | +11.5 |
| SMk19 | P02587 | Troponin C | +14.5 |
| SMk50 | Y00760 | Troponin-C | +19.6 |
| SMk57 | AAA91854 | Troponin-C | +14.6 |
| SM1535 | P02554 | Tubulin beta chain | +2.8 |
| SM1063 | P20152 | Vimentin | −5.4 |

Metabolism

| | | | |
|---|---|---|---|
| SMk56 | AAA37210 | Aldolase A | +5.5 |
| SM995 | CAA59331 | Carbonate dehydratase | +3.2 |
| SMk344 | NM_012839 | Cytochrome C | +3.4 |
| SM800 | AAG53955 | Cytochrome c oxidase subunit I | +3.0 |
| SM51 | T10974 | Cytochrome-c oxidase | +3.8 |
| SMk151 | CAA06313 | Fructose-1,6-bisphosphatase | +7.1 |
| SM2070 | P00339 | L-lactate dehydrogenase M chain | +12.7 |
| SMk120 | AJ275968 | LIM domains 1 protein | +8.6 |
| SMk147 | X59418 | NADH dehydrogenase | +2.4 |
| SM928 | O79874 | NADH-ubiquinone oxidoreductase | +5.3 |
| | | chain 1 | |
| SMk18 | AAG28185 | NADH4L | +2.1 |
| SMk81 | O19094 | Octanoyltransferase(COT) | +3.2 |
| SM295 | AB006852 | Phosphoarginine phosphatase | +2.6 |
| SMk346 | M97664 | Phosphoglucomutase isoform 2 mRNA | +5.5 |
| SM36 | TVMVRR | Protein-tyrosine kinase | +4.3 |
| SM887 | P11980 | Pyruvate kinase | +8.5 |
| SM698 | S64635 | Pyruvate kinase | +9.7 |
| SM723 | P52480 | Pyruvate kinase | +7.3 |
| SMk79 | U44751 | Pyruvate kinase | +5.2 |
| SMk135 | Z98820 | Sarcolipin | +3.0 |
| SM1033 | XM_018138 | Tyrosine phosphatase type IVA | +2.9 |
| SMk347 | X99312 | UDP glucose pyrophosphorylase | +3.0 |

Gene/protein expression

| | | | |
|---|---|---|---|
| SM75 | U09823 | Elongation factor 1 alpha | −4.3 |
| SM1989 | AAH05660 | Elongation factor 1 alpha 1 | −3.9 |
| SMk61 | NP_031959 | Enolase 3 | +3.6 |
| SM968 | Y00104 | Repetitive dna sequence element | −2.5 |
| | | RPE-1 | |
| SMk91 | AAC48501 | Reticulum protein | +4.6 |
| SM2083 | NP_003083 | Ribonucleoprotein polypeptide B | +3.1 |
| SM896 | AAH01127 | Ribosomal protein | +2.0 |
| SM1668 | AAH07512 | Ribosomal protein L18a | +2.1 |
| SM1784 | 228176 | Ribosomal protein P0 | +6.2 |
| SM1801 | AAA30799 | Transfer RNA-Trp synthetase | +6.0 |
| SM997 | 51077272 | Translation initiation factor | +3.5 |
| | | eif1 | |

Cell signaling/communication

| | | | |
|---|---|---|---|
| SM464 | AJ002189 | Complete mitochondrial DNA | +3.9 |
| SM732 | AF304203 | Mitochondrion | +5.9 |
| SMk11 | XM_006515 | Potassium channel | −2.4 |
| SMk187 | BC007462 | Similar to creatine kinase | +3.5 |

Cell division

| | | | |
|---|---|---|---|
| SM1067 | XP_007399 | Protease, cysteine, 1 | +3.1 |

Immune response

| | | | |
|---|---|---|---|
| SM154 | AF036005 | Interleukin-2 receptor alpha | −2.5 |
| | | chain | |
| SMk1 | AAAG52886 | Kel-like protein | +6.4 |
| SM401 | AJ251829 | MHC class I SLA genomic region | −3.0 |

EST

| | | | |
|---|---|---|---|
| SM824 | AK023385 | cDNA FLJ13323 fis | +2.5 |
| SM1776 | XM_050494 | KIAA0182 protein | +3.6 |
| SM1556 | XP_043678 | KIAA1096 protein | +4.9 |

Unknown

| | | | |
|---|---|---|---|
| SM1785 | AC015998 | AC015998 | +2.1 |
| SM2152 | BI327422 | AR078G01iTHYEG01S | −4.0 |
| SM1469 | BG938561 | Cn26h08.x1 | −2.2 |
| SM908 | AAG28205 | COI | +2.8 |
| SM851 | AAG28192 | COI | +3.6 |
| SM1738 | CAA19420 | DJ466P17.1.1(Laforin) | +4.8 |
| SM1007 | AAD31021 | Foocen-m | +3.8 |
| SM1920 | BE421626 | HWM012cA.1 | +3.3 |
| SM1972 | XP_039195 | Hypothetical protein | +3.2 |
| SM1536 | T08758 | Hypothetical protein | +4.7 |
| SMk137 | XP_002275 | Hypothetical protein | +20.0 |
| SM1724 | XP_016035 | Hypothetical protein | −2.6 |
| SM1539 | AT001097 | Mandarina library | −2.3 |
| SM1474 | BG384994 | MARC 1PI | +2.6 |
| SM1853 | BF198401 | MARC 2PIG | +3.6 |
| SM1941 | BE925069 | MR1-AN0039-290800-004-a01 | +4.4 |
| SM379 | AW328623 | NIH_MGC_4 | +2.3 |
| SM1911 | BE872239 | NIH_MGC_65 | −2.4 |
| SM1676 | BG548727 | NIH_MGC_77 | +5.1 |
| SM1914 | BG534187 | NIH_MGC_77 | −2.3 |
| SM1650 | BI337009 | Peripheral Blood Cell cDNA | +9.3 |
| | | library | |
| SM1064 | BAB28119 | Putative | +3.4 |
| SM618 | BAB28422 | Putative | +2.1 |
| SM1774 | BAB30715 | Putative | +3.2 |
| SM1690 | BF864360 | Reinhardtii CC-1690 | +2.2 |
| SM1898 | F23148 | Small intestine cDNA library | −2.3 |
| SM96 | M17733 | Thymosin beta-4 mRNA | −4.2 |
| SM1922 | AAH03026 | Unknown | +4.0 |
| SM210 | BAA91923 | Unnamed protein product | −3.1 |

No match

| | | |
|---|---|---|
| SM107 | No match | −2.4 |
| SM278 | No match | −2.2 |
| SM384 | No match | −2.3 |
| SMk37 | No match | +7.7 |
| SM717 | No match | −3.0 |
| SM1598 | No match | +4.5 |
| SMk6 | No match | +3.8 |
| SMk68 | No match | +5.0 |
| SM1100 | No match | −2.6 |
| SMk70 | No match | +3.9 |
| SMk80 | No match | +17.7 |
| SMk112 | No match | +3.5 |
| SM1639 | No match | −4.0 |
| SMk148 | No match | +3.8 |
| SM1665 | No match | +3.8 |
| SM1665 | No match | +13.0 |
| SMk95 | No match | +2.7 |
| SMk133 | No match | +2.4 |
| SMk152 | No match | +6.4 |
| SM1897 | No match | +3.4 |
| SMk138 | No match | +10.3 |
| SM1902 | No match | +2.1 |
| SMk342 | No match | +6.7 |
| SMk181 | No match | +11.0 |
| SM904 | No match | −3.4 |
| SMk262 | No match | +3.9 |
| SM9 | No match | +2.4 |
| SM1964 | No match | +2.6 |
| SMk335 | No match | −3.9 |

†agreed Accession no.
**Information agreed to the database
No match: No information agreed to the database; novel EST
ESM: early stage muscle (body weight 30 kg),
ASM: adult stage muscle (body weight 90 kg),
SM: swine muscle

As shown in Table 1, 14 genes which are expressed in ASM, identified in Table 1 and known for their functions have not yet precisely measured. These genes include actin alpha 1, tropomyosin alpha chain, aldolase A, fructose-1,6-bisphosphatase, NADH-ubiquinone oxidoreductase chain 1, phosphoglucomutase isoform 1 mRNA, pyruvate kinase, mitochondrion, kel-like proteins (Table 2). Actin cytoskeleton comprising microfilaments is responsible for various functions in eukaryotic cells including intracellular transport and structure support. Actin exists in the form of a monomer (G-actin) or filament (F-actin). The F-actin is a main component of the microfilament. Many proteins regulate the length, location and transform of the microfilament. The actin cytoskeleton has a variable structure which can immediately change the shape and structure in response to a stimulus and in the course of the cell cycle. The structure of the actin cytoskeleton is not fixed but varied in response to the cellular environment. Tropomyosin with troponin complexes (troponin-I, -T and C) bonded thereto plays an important role in Ca²⁺ dependent regulation upon contraction of linear muscle in vertebrata. Tropomyosin is closely connected to a protein group having an alpha coiled coil structure comprising a dimmer. Pyruvate kinase which catalyzes transphosphorylation of PEP to ADP in mammals is one of the important regulation enzymes and its property to regulate the metabolic pathways is closely involved in various metabolic demands needed in other tissues during pathway regulation. Thus, the present inventors use it as an object of study.

Also, 5 genes which are expressed in ESM, identified in Table 1 and Table 2 and not known for their functions have not yet precisely measured. These genes include collagen, disk/large homologue 5 (fruit fly), acid secret proteins, vimentin. Collagen is a main component of extracellular matrix and comprises at least 18 types of different macro protein groups, which are observed upon cell division, replication, migration and attachment in the course of embryo development and various morphological differentiations and partially regulated by the cellular interaction of surrounding extracellular matrix.

The expression of vimentin coding genes (Vim) is one of the terminal markers which appear after a serial of genetic events occurring in the course of differentiation of leukocyte to macrophage. Therefore, valuation of transcriptional regulation mechanism is an important stage to understand the genetic regulation pathways responsible for the leukocyte differentiation.

TABLE 2
Expression ratio of differentially expressed genes
between ESM and ESF

| | | | |
|---|---|---|---|
| | | | Ratio of |
| ESTs | Accession | | gene expression |
| No. | No†. | Description** | ESF(30)/ESM(30) |

Cellular structure and motility

| | | | |
|---|---|---|---|
| SM2149 | CAB56598 | 1-alpha dynein heavy chain | −2.1 |
| SM781 | NP_033891 | 19 kDa-interacting protein 3- | +2.2 |
| | | like | |
| SM1068 | AAF20165 | Actin | +4.5 |
| SM635 | BAB19361 | Actin | +2.6 |
| SM106 | P53506 | Actin | +4.9 |
| SM768 | X52815 | Actin | +2.4 |
| SM363 | B25819 | Actin | +3.7 |
| SM713 | AAA51586 | Actin | +5.6 |
| SMk77 | NM_001100 | Actin, alpha 1 | +4.5 |
| SM128 | NP_033740 | Actin, gamma 2 | +3.9 |
| SM1091 | JC5971 | Alpha-b crystallin | +2.1 |
| SM902 | BC001748 | Annexin A2 | −4.2 |
| SM846 | P81287 | Annexin V | −3.5 |
| SM653 | P04272 | Annexin II | −2.3 |
| SMk340 | U75316 | Beta-myosin heavy chain mRNA | +2.2 |
| SM1807 | AAF99682 | Calpain large polypeptide L2 | +2.7 |
| SM541 | NP_000079 | Collagen | −4.9 |
| SM715 | L47641 | Collagen | −5.2 |
| SM1023 | Q9XSJ7 | Collagen alpha 1 | −4.6 |
| SM758 | CGHU1S | Collagen alpha 1 | −4.3 |
| SM62 | CGHU2V | Collagen alpha 2 | −4.4 |
| SM949 | O46392 | Collagen alpha 2 | −3.2 |
| SM410 | CAA28454 | Collagen(alpha V) | −2.3 |
| SM1121 | NM_000393 | Collagen, type V, alpha 2 | −2.8 |
| SM53 | NP_000384 | Collagen, type V, alpha 2 | −2.5 |
| SM1651 | XM_039583 | Discs, large(Drosophila) | −8.6 |
| | | homolog 5 | |
| SM1050 | AAA30521 | Fibronectin | −3.1 |
| SM381 | FNHU | Fibronectin precursor | −2.6 |
| SM122 | P07589 | Fibronectin(FN) | −2.5 |
| SM1573 | XM_044160 | Lamin A/C | +2.1 |
| SMk55 | NP_006462 | Myosin | +3.6 |
| SMk168 | AB025261 | Myosin heavy chain | +5.0 |
| SM1732 | NP_004678 | Myotubularin related protein 4 | +4.7 |
| SM690 | NP_003109 | Secreted protein, acidic | −5.2 |
| SM1043 | P06469 | Tropomyosin alpha chain | +8.6 |
| SMk173 | X66274 | Tropomysin | +2.2 |
| SMk19 | P02587 | Troponin C | +6.9 |
| SMk57 | AAA91854 | Troponin-C | +7.1 |
| SMk50 | Y00760 | Troponin-C | +9.0 |
| SM1535 | P02554 | Tubulin beta chain | +3.3 |
| SM1063 | P20152 | Vimentin | −5.1 |
| SM730 | CAA69019 | Vimentin | −3.2 |

Metabolism

| | | | |
|---|---|---|---|
| SMk344 | NM_012839 | Cytochrome C | +2.4 |
| SM800 | AAG53955 | Cytochrome c oxidase subunit I | +2.9 |
| SMk151 | CAA06313 | Fructose-1,6-bisphosphatase | +4.2 |
| SMk254 | 231300 | Glycogen Phosphorylase b | +2.6 |
| SM2070 | P00339 | L-lactate dehydrogenase M chain | +10.6 |
| SM928 | O79874 | NADH-ubiquinone oxidoreductase | +3.2 |
| | | chain 1 | |
| SMk81 | O19094 | Octanoyltransferase(COT) | +3.9 |
| SM295 | AB006852 | Phosphoarginine phosphatase | +2.3 |
| SMk346 | M97664 | Phosphoglucomutase isoform 2 mRNA | +3.3 |
| SM36 | TVMVRR | Protein-tyrosine kinase | +2.6 |
| SM723 | P52480 | Pyruvate kinase | +7.5 |
| SM698 | S64635 | Pyruvate kinase | +6.6 |
| SM887 | P11980 | Pyruvate kinase | +6.3 |
| SM1594 | AAA62278 | Superoxide dismutase | −3.2 |
| SM1033 | XM_018138 | Tyrosine phosphatase type IVA | +2.2 |

Gene/protein expression

| | | | |
|---|---|---|---|
| SM75 | U09823 | Elongation factor 1 alpha | −3.7 |
| SM1989 | AAH05660 | Elongation factor 1 alpha 1 | −3.8 |
| SMk120 | AJ275968 | LIM domains 1 protein | +9.9 |
| SMk91 | AAC48501 | Reticulum protein | +2.1 |
| SM2083 | NP_003083 | Ribonucleoprotein polypeptide B | +3.2 |
| SM21 | NP_000994 | Ribosomal | +2.2 |
| SM1784 | 228176 | Ribosomal protein P0 | +5.5 |
| SM1820 | BC014277 | Tissue inhibitor of | −2.6 |
| | | metalloproteinase 3 | |
| SM1801 | AAA30799 | Transfer RNA-Trp synthetase | +5.7 |
| SM997 | 51077272 | Translation initiation factor | +2.3 |
| | | eif1 | |

Cell signaling/communication

| | | | |
|---|---|---|---|
| SM464 | AJ002189 | Complete mitochondrial DNA | +2.7 |

Immune response

| | | | |
|---|---|---|---|
| SMk1 | AAG52886 | Kel-like protein 23 | +4.6 |

EST

| | | | |
|---|---|---|---|
| SM1776 | XM_050494 | KIAA0182 | +3.2 |
| SM1556 | XP_043678 | KIAA1096 protein | +4.5 |

Unknown

| | | | |
|---|---|---|---|
| SM2152 | BI327422 | AR078G01iTHYEG01S | −5.5 |
| SMk3 | AL13277 | Chromosome 14 DNA sequence | +2.3 |
| SM908 | AAG28205 | COI | +2.2 |
| SM1738 | CAA19420 | DJ466P17.1.1(Laforin) | +3.5 |
| SM1007 | AAD31021 | Foocen-m | +3.0 |
| SM1724 | XP_016035 | Hypothetical protein | −2.6 |
| SMk137 | XP_002275 | Hypothetical protein | +10.0 |
| SM1972 | XP_039195 | Hypothetical protein | +2.8 |
| SM787 | AF192528 | Integrin beta-1 subunit | +2.0 |
| SM1474 | BG384994 | MARC 1PI | +2.8 |
| SM1676 | BG548727 | NIH_MGC_77 | +2.3 |
| SM1650 | BI337009 | Peripheral Blood Cell cDNA | +7.3 |
| | | library | |
| SM1774 | BAB30715 | Putative | +5.1 |
| SM1064 | BAB28119 | Putative | +3.0 |
| SM1690 | BF864360 | Reinhardtii CC-1690 | +2.5 |
| SM96 | M17733 | Thymosin beta-4 mRNA | −3.9 |
| SM1922 | AAH03026 | Unknown | +4.7 |

No match

| | | |
|---|---|---|
| SMk58 | No match | +2.9 |
| SM717 | No match | −4.4 |
| SMk6 | No match | +2.4 |
| SMk68 | No match | +3.2 |
| SMk80 | No match | +4.3 |
| SMk112 | No match | +2.1 |
| SM1639 | No match | −2.8 |
| SMk148 | No match | +2.9 |
| SM1665 | No match | +9.8 |
| SMk95 | No match | +2.1 |
| SMk152 | No match | +6.4 |
| SM1897 | No match | +2.6 |
| SMk138 | No match | +3.1 |
| SM796 | No match | −2.2 |
| SMk342 | No match | +3.9 |
| SMk181 | No match | +4.4 |
| SM904 | No match | −2.7 |
| SMk262 | No match | +2.7 |
| SM9 | No match | +2.9 |
| SM1964 | No match | +2.6 |
| SMk335 | No match | +3.8 |

†agreed Accession no.
**Information agreed to the database
No match: No information agreed to the database; novel EST
ESM: early stage muscle (body weight 30 kg),
ESF: early stage fat (body weight 30 kg),
SM: swine muscle

As shown in Table 2, 13 genes include expressed in ESF include troponin -C, L-lactate dehydrogenase M chain, LIM domain 1 protein, pyruvate kinase, ribosome protein P0, transfer RNA-Trp syntase. The genome clones comprising human pyruvate kinase M(PKM) genes encoding M1 type and M2 type isozyme were isolated and measured for their exon sequences. The genes were about 32 kb and comprise 12 exons and 11 introns. The exon 9 and 10 comprise sequences specific to the M1 type and M2 type, respectively, which indicates that the human isozyme is produced from the same gene by selective splicing, like the genes of rat. 4½LIM domain protein 1 (FHL1) was initially used as an abundant skeletal muscle protein having 4 LIM domains and 1 GATA such as zinc finger. FHL1 was shown to be expressed in the skeletal muscle as well as various tissues. In recent, it has been identified that selectively inserted FHL1 mRNA encodes proteins with the C-end deleted. It was found that FHLlC ultimately produces N-end comprising 16 amino acids in the skeletal muscle of sine by a newly identified initiation codon. From the above results, these genes were evaluated as meat quality-related candidate genes.

Thus, the expression rate was 2 times more for genes identified in ESM vs ASM and ESM vs ESF. By cDNA microarray analysis, total 128 genes which had been significantly over-expressed were identified. Actin, beta-myosin, glycogen phosphorylase, myosin heavy chain, novel genes, pyruvate kinase, troponin C were specifically expressed in ESM. collagen, fibronectin, an inhibitor of metalloproteinase 3, intergrin beta-1 subunit were specifically expressed in ESF. 1-alpha dynein heavy chain, 601446467F1, assumed protein, fibronectin precursor, MHC class I, novel genes, anonymous protein products were specifically expressed in ASM. These genes were evaluated as meat quality-related candidate genes. Also, the present inventors, from now on, will conduct research on functions of more genes to bring a high meat quality swine.

### Example 2

### Construction of the Inventive Functional cDNA Chip for Meat Quality Evaluation and Screening of Specific Genes in Swine

The fat specific genes according to the growth stages in swine, screened in Example 1, including the collagen, fibronectin, inhibitor of metalloproteinase 3 and integrin β-1 subunit were immobilized on a DNA microarray and fabricated into a functional cDNA chip for meat quality evaluation and screening of specific genes in swine by the method of Preparation Example 1.

### Example 3

### Construction of the Inventive Kit for Meat Quality Evaluation and Screening of Specific Genes in Swine

A kit for meat quality evaluation and screening of specific genes in swine comprising the functional cDNA chip fabricated in Example 2, Cy5-dCTP or Cy3-dCTP bound cDNA from RNA of the tissue to be screened, a fluorescence scanning system and a computer analysis system was fabricated.

### Industrial Applicability

As explained through the Examples, the present invention relates to screening of the expression profile of fat specific genes according to the growing stages in swine and a functional cDNA chip using the same and provides expression files of the fat specific genes specifically expressed according to the growing stages in the muscle and fat tissues of swine. Also, the present invention provides a functional cDNA chip for meat quality evaluation and screening of specific genes in swine prepared by integrating only the fat specific genes screened as described above. Therefore, the functional cDNA chip can be used to evaluate of meat quality according to breeds of swine and to bring a high meat quality swine, thereby being very useful for the hog raising industry.

1. A functional cDNA chip for meat quality evaluation and screening of specific genes comprising a probe comprising fat specific genes specifically expressed in the muscle and fat tissues of swine and a substrate on which the probe is immobilized.

2. The functional cDNA chip according to claim 1, wherein the probe DNA includes collagen, fibronectin, inhibitor of metalloproteinase 3 and integrin β-1 subunit.

3. A kit for meat quality evaluation and screening of specific genes in swine comprising the functional cDNA chip having fat specific genes according to the growing stages in swine, as defined in claim 1, integrated thereon, Cy5-dCTP or Cy3-dCTP bound cDNA from RNA of the tissue to be screened, a fluorescence scanning system and a computer analysis system
